(19) United States
(12) Patent Application Publication
(10) Pub. No.: US 2020/0184638 A1
(43) Pub. Date: Jun. 11, 2020

(54) SYSTEMS AND METHODS FOR ENHANCING SURGICAL IMAGES AND/OR VIDEO

(71) Applicant: Covidien LP, Mansfield, MA (US)

(72) Inventors: Dwight Meglan, Westwood, MA (US); Meir Rosenberg, Newton, MA (US)

(21) Appl. No.: 16/638,819

(22) PCT Filed: Aug. 16, 2018
(86) PCT No.: PCT/US2018/000292

§ 371 Date: Feb. 13, 2020

Related U.S. Application Data
(60) Provisional application No. 62/546,169, filed on Aug. 16, 2017.

(51) Int. Cl. G06T 7/00; G06T 5/10; A61B 90/00; A61B 34/10

(52) U.S. Cl. CPC G06T 7/0012; G06T 5/10; A61B 90/361; A61B 34/10; A61B 34/35; G06T 2207/10016; G06T 2207/10068; G06T 2207/30004; G06T 2207/20182

(57) ABSTRACT

A system for enhancing an image during a surgical procedure includes an image capture device configured to be inserted into a patient and capture an image inside the patient. The system also includes a controller that applies at least one image processing filter to the image to generate an enhanced image. The image processing filter includes a spatial decomposition filter that decomposes the image into a plurality of spatial frequency bands, a frequency filter that filters the plurality of spatial frequency bands to generate a plurality of filtered enhanced bands, and a recombination filter that generates the enhanced image to be displayed by a display.

### BACKGROUND

Minimally invasive surgeries involve the use of multiple small incisions to perform a surgical procedure instead of one larger opening or incision. The small incisions have reduced patient discomfort and improved recovery times. The small incisions have also limited the visibility of internal organs, tissue, and other matter.

Endoscopes have been used and inserted in one or more of the incisions to make it possible for clinicians to see internal organs, tissue, and other matter inside the body during surgery. When performing an electrosurgical procedure during a minimally invasive surgery, it is not uncommon for a clinician to see smoke arising from vaporized tissue thereby temporarily obscuring the view provided by the endoscope. Conventional methods to remove smoke from the endoscopic view include evacuating air from the surgical environment.

There is a need for improved methods of providing a clinician with an endoscopic view that is not obscured by smoke.

### SUMMARY

The present disclosure relates to surgical techniques to improve surgical outcomes for a patient, and more specifically, to systems and methods for removing temporary obstructions from a clinician's field of vision while performing a surgical technique.

In an aspect of the present disclosure, a system for enhancing a surgical image during a surgical procedure is provided. The system includes an image capture device configured to be inserted into a patient and capture an image inside the patient during the surgical procedure and a controller configured to receive the image and apply at least one image processing filter to the image to generate an enhanced image. The image processing filter includes a spatial and/or temporal decomposition filter and a recombination filter. The spatial decomposition filter is configured to decompose the image into a plurality of spatial frequency bands, a frequency filter is configured to filter the plurality of spatial frequency bands to generate a plurality of enhanced bands. The recombination filter is configured to generate the enhanced image by collapsing the plurality of enhanced bands. The system also includes a display configured to display the enhanced image to a user during the surgical procedure.

In some embodiments, the image capture device captures a video having a plurality of images and the controller applies the at least one image processing filter to each image of the plurality of images.

In some embodiments, the frequency filter is a temporal filter. In other embodiments, the frequency filter is a color filter. The frequency of the frequency filter may be set by a clinician or by an algorithm based on objective functions such as attenuating the presence of a band of colors whose movement fits with in a spatial frequency band.

In another aspect of the present disclosure, a method for enhancing at least one image during a surgical procedure is provided. The method includes capturing at least one image using an image capture device and filtering the at least one image. Filtering the at least one image includes decomposing the at least one image to generate a plurality of spatial frequency bands, applying a frequency filter to the plurality of spatial frequency bands to generate a plurality of enhanced bands, and collapsing the plurality of enhanced bands to generate the enhanced image. The method also includes displaying the enhanced image on a display.

In some embodiments, the method also includes capturing a video having a plurality of images and filtering each image of the plurality of images.

In some embodiments, the enhanced filter is a temporal filter. In other embodiments, the enhanced filter is a color filter. The frequency of the enhanced filter may be set by a clinician.

In some embodiments, applying the frequency filter to the plurality of spatial frequency bands to generate the plurality of enhanced bands includes applying a color filter to the plurality of spatial frequency bands to generate a plurality of partially enhanced bands and applying a temporal filter to the plurality of partially enhanced bands to generate the plurality of enhanced bands. Applying the color filter to the plurality of spatial frequency bands to generate the plurality of partially enhanced bands can include isolating an obstruction to a portion of the partially enhanced bands. Applying the temporal filter to the plurality of partially enhanced bands to generate the plurality of enhanced bands may include applying the temporal filter only to the portion plurality of partially enhanced bands including the obstruction.

Further, to the extent consistent, any of the aspects described herein may be used in conjunction with any or all of the other aspects described herein.

### BRIEF DESCRIPTION OF THE DRAWINGS

The above and other aspects, features, and advantages of the present disclosure will become more apparent in light of the following detailed description when taken in conjunction with the accompanying drawings in which:

FIG. 1 is a block diagram of a system for enhancing a surgical environment in accordance with an embodiment of the present disclosure;

FIG. 2 is a system block diagram of the controller of FIG. 1;

FIG. 3 is a block diagram of a system for enhancing an image or video in accordance with an embodiment of the present disclosure;

FIG. 4 is a block diagram of a system for enhancing an image or video in accordance with another embodiment of the present disclosure;

FIG. 5 shows an example of a captured image and an enhanced image; and

FIG. 6 is a system block diagram of a robotic surgical system in accordance with an embodiment of the present disclosure.

### DETAILED DESCRIPTION

Image data captured from an endoscope during a surgical procedure may be analyzed to detect color changes or movement within the endoscope's field of view. Various image processing technologies may be applied to this image data to identify and enhance or decrease these color changes and/or movements. For example, Eulerian image amplification/minimization techniques may be used to identify and modify “color” changes of light in different parts of a displayed image.

Phase-based motion amplification techniques may also be used to identify motion or movement across image frames. In some instances, changes in a measured intensity of predetermined wavelengths of light across image frames may be presented to a clinician to make the clinician more aware of the motion of particular objects of interest.

Eulerian image amplification and/or phase-based motion amplification technologies may be included as part of an imaging system. These technologies may enable the imaging system to provide higher detail for a specific location within an endoscope's field of view.

One or more of these technologies may be included as part of an imaging system in a surgical robotic system to provide a clinician with additional information within an endoscope's field of view. This may enable the clinician to quickly identify, avoid, and/or correct undesirable situations and conditions during surgery.

The present disclosure is directed to systems and methods for providing enhanced images in real time to a clinician during a surgical procedure. The systems and methods described herein apply image processing filters to a captured image to provide an image free of obscurities. In some embodiments, the systems and methods permit video capture during a surgical procedure. The captured video is processed in real time or near real time and then displayed to the clinician as an enhanced image. The image processing filters are applied to each frame of the captured video. Providing the enhanced image or video to the clinician provides the clinician with an unobscured view.

The embodiments described herein enable a clinician to view a region of interest with sufficient detail to ensure the effectiveness of a surgical procedure.

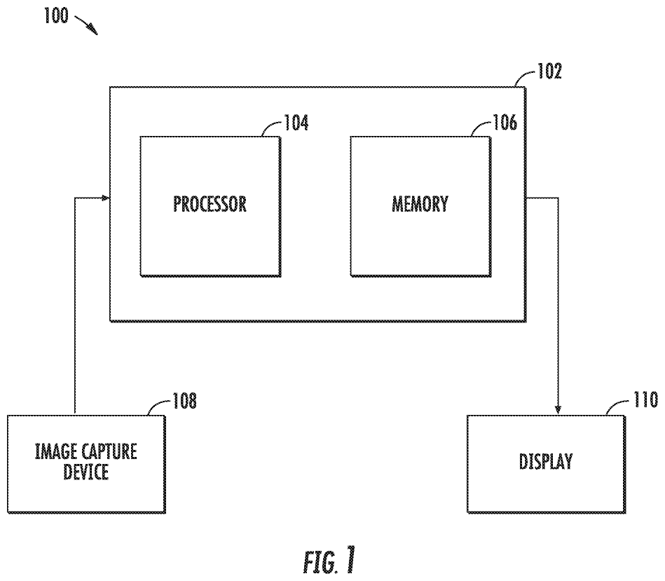

Turning to FIG. 1, a system for enhancing images and/or video of a surgical environment, according to embodiments of the present disclosure, is shown generally as 100. System 100 includes a controller 102 that has a processor 104 and a memory 106. The system 100 also includes an image capture device 108, e.g., a camera, that records still frame images or moving images. Image capture device 108 may be incorporated into an endoscope, stereo endoscope, or any other surgical toll that is used in minimally invasive surgery. A display 110, displays enhanced images to a clinician during a surgical procedure. Display 110 may be a monitor, a projector, or a pair of glasses worn by the clinician. In some embodiments, the controller 102 may communicate with a central server (not shown) via a wireless or wired connection. The central server may store images of a patient or multiple patients that may be obtained using x-ray, a computed tomography scan, or magnetic resonance imaging.

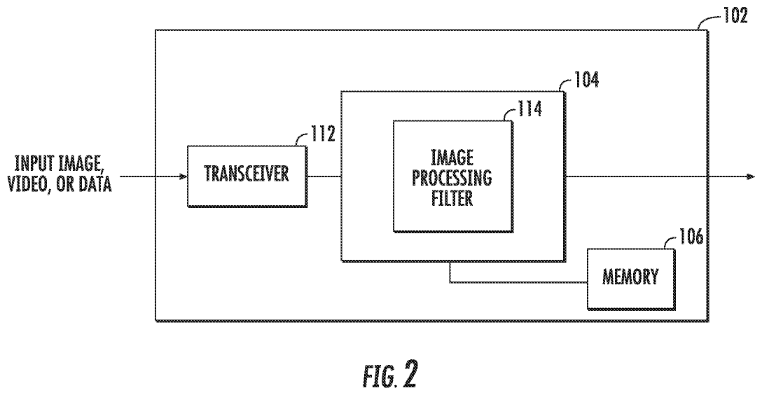

FIG. 2 depicts a system block diagram of the controller 102. As shown in FIG. 2, the controller 102 includes a transceiver 112 configured to receive still frame images or video from the image capture device 108. In some embodiments, the transceiver 112 may include an antenna to receive the still frame images, video, or data via a wireless communication protocol. The still frame images, video, or data are provided to the processor 104. The processor 104 includes an image processing filter 114 that processes the received still frame images, video, or data to generate an enhanced image or video. The image processing filter 114 may be implemented using discrete components, software, or a combination thereof. The enhanced image or video is provided to the display 110.

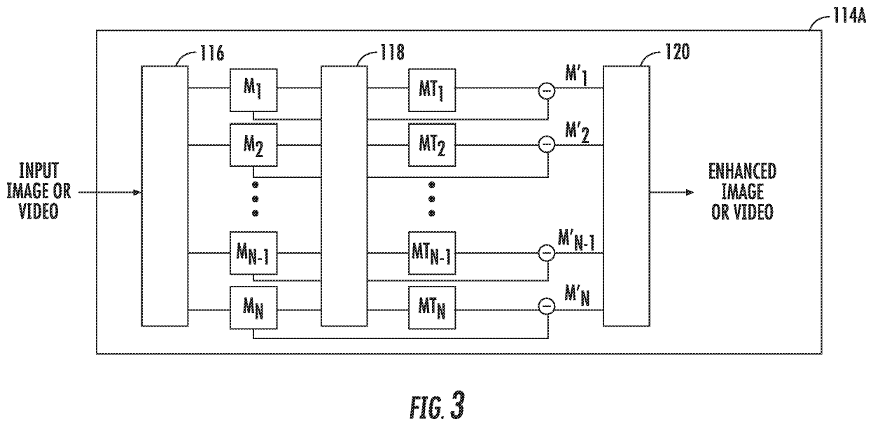

Turning to FIG. 3, a system block diagram of a motion filter that may be applied to images and/or video received by transceiver 112 is shown as 114A. The motion filter 114A is one of the filters included in image processing filter 114. In the motion filter 114A, each frame of a received video is decomposed into different spatial frequency bands M₁ to M_{N} using a spatial decomposition filter 116. The spatial decomposition filter 116 uses an image processing technique known as a pyramid in which an image is subjected to repeated spatial filters that yield a selectable number of levels (constrained by sampling size of the image) each of which consists of differing maximum values of spatial frequency related information. The spatial filters specifically designed to enable unique pyramid levels of varying frequency content to be constructed.

After the frame is subjected to the spatial decomposition filter 116, a frequency or temporal filter 118 is applied to all the spatial frequency bands M₁ to M_{N} to generate temporally filtered bands MT₁ to MT_{N}. The temporal filter 118 can be a bandpass filter or a band-stop filter that is used to extract one or more desired frequency bands. For example, if the clinician is performing an electrosurgical procedure and wants to eliminate smoke from the images, a band-stop or notch filter may be set by the clinician to a frequency that corresponds to the movement of smoke. In other words, the notch filter is set to a narrow range that includes the movement of smoke and applied to all the spatial frequency bands M₁ to M_{N}. It is envisioned that the frequency of the notch filter can be set based on an obstruction to be removed or minimized from the frame. The spatial frequency band that corresponds to the set range of the notch filter is attenuated to enhance all of the temporally filtered bands MT₁ to MT_{N} from the original spatial frequency bands M₁ to M_{N} to generate enhanced bands M′₁ to M′_{N}.

Each frame of the video is then reconstructed using a recombination filter 70 by collapsing enhanced bands M′₁ to M′_{N} to generate an enhanced frame. All the enhanced frames are combined to produce the enhanced video. The enhanced video that is shown to the clinician includes the surgical environment without the obstruction, e.g., smoke.

In some embodiments, a color filter 114B (e.g., a color amplification filter) may be applied before using a motion filter (e.g., motion filter 114A) to improve the enhanced image or video. By setting the color filter 114B to a specific color frequency band, removal of certain items, e.g., smoke, from the enhanced image shown on display 110 can be improved permitting the clinician to easily see the surgical environment without any obstructions. The color filter 114B may identify the obstruction in the spatial frequency band using one or more colors before a motion filter is applied. The color filter 114B may isolate obstructions to a portion of the frame allowing the motion filter to be applied to the isolated portion of the frame. By only applying the motion filter to the isolated portion of the frame, speed of the generating and displaying the enhanced image or video can be increased.

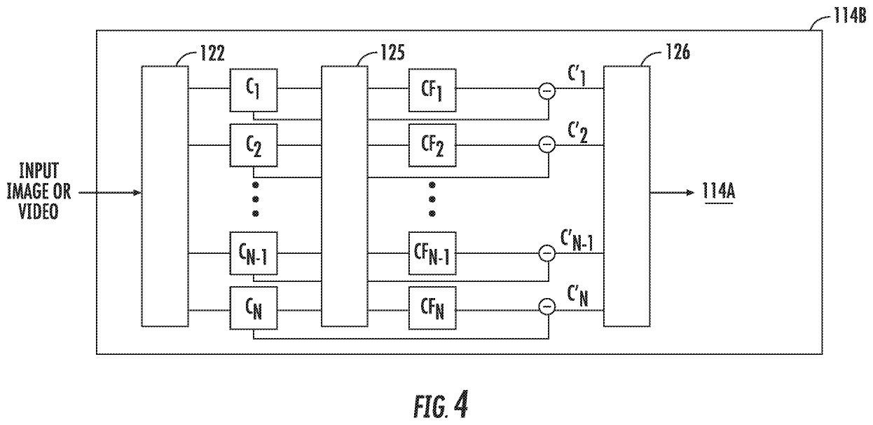

For example, FIG. 4 is a system block diagram of the color filter 114B that may be applied to images and/or video received by transceiver 112. Color filter 114B is another one of the filters included in image processing filter 114. In the color filter 114B, each frame of a received video is decomposed into different spatial frequency bands C₁ to C_{N} using a spatial decomposition filter 122. Similar to spatial decomposition filter 116, the spatial decomposition filter 122 also uses an image processing technique known as a pyramid in which an image is subjected to repeated spatial filters that yield a selectable number of levels.

After the frame is subjected to the spatial decomposition filter 122, a frequency or color filter 124 is applied to all the spatial frequency bands C₁ to C_{N} to generate color filtered bands CF₁ to CF_{N}. The color filter 124 is can be a bandpass filter or a band-stop filter that is used to extract one or more desired frequency bands. For example, if the clinician is performing an electrosurgical technique that causes smoke to emanate from vaporized tissue, a band-stop or notch filter may be set by the clinician to a frequency that corresponds to the color of the smoke. In other words, the notch filter is set to a narrow range that includes the smoke and applied to all the spatial frequency bands C₁ to C_{N}. It is envisioned that the frequency of the notch filter can be set based on an obstruction to be removed or minimized from the frame. The spatial frequency band that corresponds to the set range of the notch filter is attenuated to enhance all of the color filtered bands CF₁ to CF_{N} from the original spatial frequency bands C₁ to C_{N} to generate enhanced bands C′₁ to C′_{N}.

Each frame of the video is then reconstructed using a recombination filter 126 by collapsing enhanced bands C′₁ to C′_{N} to generate an enhanced frame. All the enhanced frames are combined to produce the enhanced video. The enhanced video that is shown to the clinician includes the surgical environment without the obstruction, e.g., smoke.

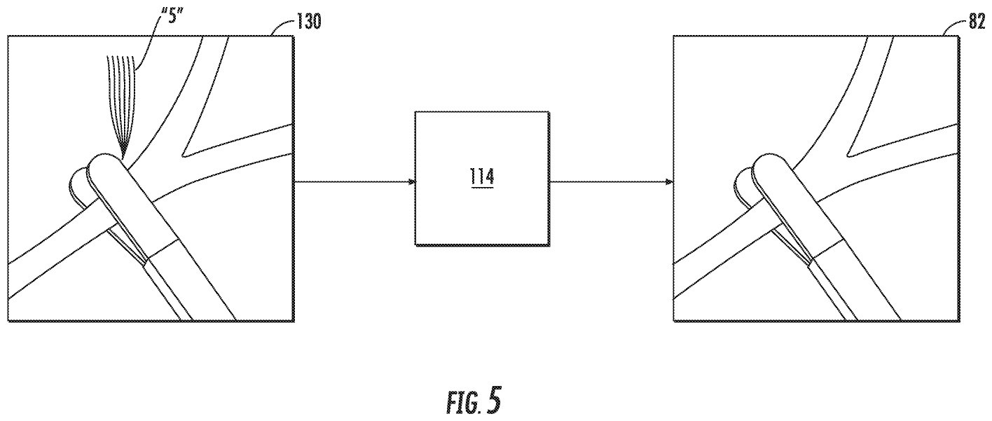

FIG. 5 depicts an image 130 of a surgical environment that is captured by the image capture device 108. Image 130 is processed by image processing filter 114, which may involve the use of motion filter 114A and/or color filter 114B, to generate an enhanced image 132. As can be seen in the enhanced image 132, the smoke “S” that was present in image 130 is removed from the enhanced image 132.

The above-described embodiments may also be configured to work with robotic surgical systems and what is commonly referred to as “Telesurgery.” Such systems employ various robotic elements to assist the clinician in the operating theater and allow remote operation (or partial remote operation) of surgical instrumentation. Various robotic arms, gears, cams, pulleys, electric and mechanical motors, etc. may be employed for this purpose and may be designed with a robotic surgical system to assist the clinician during the course of an operation or treatment. Such robotic systems may include, remotely steerable systems, automatically flexible surgical systems, remotely flexible surgical systems, remotely articulating surgical systems, wireless surgical systems, modular or selectively configurable remotely operated surgical systems, etc.

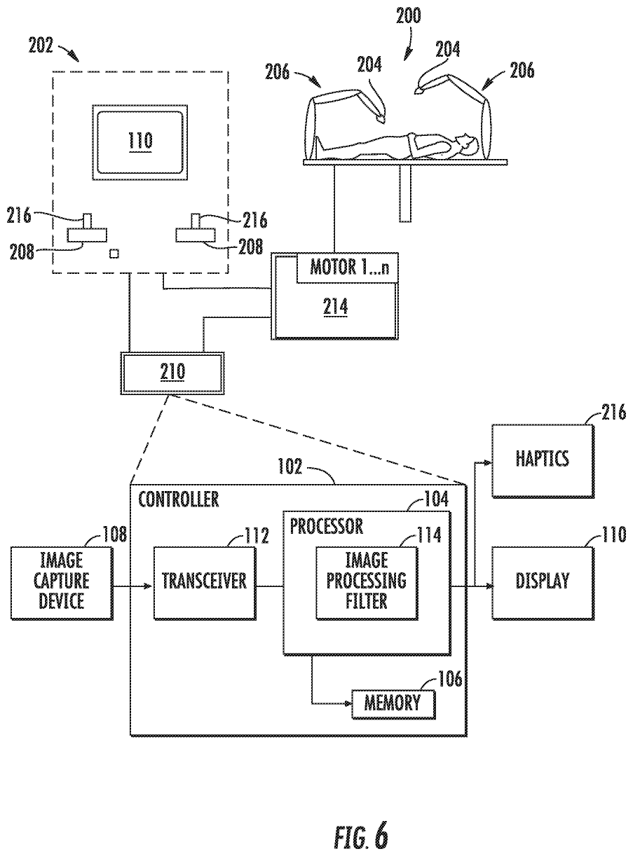

As shown in FIG. 6, a robotic surgical system 200 may be employed with one or more consoles 202 that are next to the operating theater or located in a remote location. In this instance, one team of clinicians or nurses may prep the patient for surgery and configure the robotic surgical system 200 with one or more instruments 204 while another clinician (or group of clinicians) remotely controls the instruments via the robotic surgical system. As can be appreciated, a highly skilled clinician may perform multiple operations in multiple locations without leaving his/her remote console which can be both economically advantageous and a benefit to the patient or a series of patients.

The robotic arms 206 of the surgical system 200 are typically coupled to a pair of master handles 208 by a controller 210. Controller 210 may be integrated with the console 202 or provided as a standalone device within the operating theater. The handles 206 can be moved by the clinician to produce a corresponding movement of the working ends of any type of surgical instrument 204 (e.g., probe, end effectors, graspers, knifes, scissors, etc.) attached to the robotic arms 206. For example, surgical instrument 204 may be a probe that includes an image capture device. The probe is inserted into a patient in order to capture an image of a region of interest inside the patient during a surgical procedure. One or more of the image processing filters 114A or 114B are applied to the captured image by the controller 210 before the image is displayed to the clinician on a display 110.

The movement of the master handles 208 may be scaled so that the working ends have a corresponding movement that is different, smaller or larger, than the movement performed by the operating hands of the clinician. The scale factor or gearing ratio may be adjustable so that the operator can control the resolution of the working ends of the surgical instrument(s) 204.

During operation of the surgical system 200, the master handles 208 are operated by a clinician to produce a corresponding movement of the robotic arms 206 and/or surgical instruments 204. The master handles 208 provide a signal to the controller 210 which then provides a corresponding signal to one or more drive motors 214. The one or more drive motors 214 are coupled to the robotic arms 206 in order to move the robotic arms 206 and/or surgical instruments 204.

The master handles 208 may include various haptics 216 to provide feedback to the clinician relating to various tissue parameters or conditions, e.g., tissue resistance due to manipulation, cutting or otherwise treating, pressure by the instrument onto the tissue, tissue temperature, tissue impedance, etc. As can be appreciated, such haptics 216 provide the clinician with enhanced tactile feedback simulating actual operating conditions. The haptics 216 may include vibratory motors, electroacitve polymers, piezoelectric devices, electrostatic devices, subsonic audio wave surface actuation devices, reverse-electrovibration, or any other device capable of providing a tactile feedback to a user. The master handles 208 may also include a variety of different actuators 218 for delicate tissue manipulation or treatment further enhancing the clinician's ability to mimic actual operating conditions.

The embodiments disclosed herein are examples of the disclosure and may be embodied in various forms. Specific structural and functional details disclosed herein are not to be interpreted as limiting, but as a basis for the claims and as a representative basis for teaching one skilled in the art to variously employ the present disclosure in virtually any appropriately detailed structure. Like reference numerals may refer to similar or identical elements throughout the description of the figures.

The phrases “in an embodiment,” “in embodiments,” “in some embodiments,” or “in other embodiments,” which may each refer to one or more of the same or different embodiments in accordance with the present disclosure. A phrase in the form “A or B” means “(A), (B), or (A and B)”. A phrase in the form “at least one of A, B, or C” means “(A), (B), (C), (A and B), (A and C), (B and C), or (A, B and C)”. A clinician may refer to a surgeon or any medical professional, such as a doctor, nurse, technician, medical assistant, or the like performing a medical procedure.

The systems described herein may also utilize one or more controllers to receive various information and transform the received information to generate an output. The controller may include any type of computing device, computational circuit, or any type of processor or processing circuit capable of executing a series of instructions that are stored in a memory. The controller may include multiple processors and/or multicore central processing units (CPUs) and may include any type of processor, such as a microprocessor, digital signal processor, microcontroller, or the like. The controller may also include a memory to store data and/or algorithms to perform a series of instructions.

Any of the herein described methods, programs, algorithms or codes may be converted to, or expressed in, a programming language or computer program. A “Programming Language” and “Computer Program” includes any language used to specify instructions to a computer, and includes (but is not limited to) these languages and their derivatives: Assembler, Basic, Batch files, BCPL, C, C+, C++, Delphi, Fortran, Java, JavaScript, Machine code, operating system command languages, Pascal, Perl, PL1, scripting languages, Visual Basic, metalanguages which themselves specify programs, and all first, second, third, fourth, and fifth generation computer languages. Also included are database and other data schemas, and any other metalanguages. No distinction is made between languages which are interpreted, compiled, or use both compiled and interpreted approaches. No distinction is also made between compiled and source versions of a program. Thus, reference to a program, where the programming language could exist in more than one state (such as source, compiled, object, or linked) is a reference to any and all such states. Reference to a program may encompass the actual instructions and/or the intent of those instructions.

Any of the herein described methods, programs, algorithms or codes may be contained on one or more machine-readable media or memory. The term “memory” may include a mechanism that provides (e.g., stores and/or transmits) information in a form readable by a machine such a processor, computer, or a digital processing device. For example, a memory may include a read only memory (ROM), random access memory (RAM), magnetic disk storage media, optical storage media, flash memory devices, or any other volatile or non-volatile memory storage device. Code or instructions contained thereon can be represented by carrier wave signals, infrared signals, digital signals, and by other like signals.

It should be understood that the foregoing description is only illustrative of the present disclosure. Various alternatives and modifications can be devised by those skilled in the art without departing from the disclosure. For instance, any of the enhanced images described herein can be combined into a single enhanced image to be displayed to a clinician. Accordingly, the present disclosure is intended to embrace all such alternatives, modifications and variances. The embodiments described with reference to the attached drawing FIGS. are presented only to demonstrate certain examples of the disclosure. Other elements, steps, methods and techniques that are insubstantially different from those described above and/or in the appended claims are also intended to be within the scope of the disclosure.

What is claimed is:

1. A system for enhancing a surgical image, the system comprising:
an image capture device configured to be inserted into a patient and capture an image inside the patient during a surgical procedure;
a controller configured to receive the image and apply at least one image processing filter to the image to generate an enhanced image, the image processing filter including:
a spatial decomposition filter configured to decompose the image into a plurality of spatial frequency bands;
a frequency filter configured to filter the plurality of spatial frequency bands to generate a plurality of enhanced bands; and
a recombination filter configured to generate the enhanced image by collapsing the plurality of enhanced bands; and
a display configured to display the enhanced image to a user during the surgical procedure.

2. The system of claim 1, wherein the image capture device captures a video having a plurality of images and the controller applies the at least one image processing filter to each image of the plurality of images.

3. The system of claim 1, wherein the frequency filter is a temporal filter.

4. The system of claim 1, wherein the frequency filter is a color filter.

5. The system of claim 1, wherein a frequency of the frequency filter is set by a clinician.

6. The system of claim 1, wherein a frequency of the frequency filter is preset based on a type of obstruction.

7. The system of claim 6, wherein the frequency of the frequency filter is selectable based upon a type of obstruction.

8. A method for enhancing an image during a surgical procedure, the method comprising:
capturing at least one image using an image capture device;
filtering the at least one image, wherein filtering includes:
decomposing the at least one image to generate a plurality of spatial frequency bands;
applying a frequency filter to the plurality of spatial frequency bands to generate a plurality of enhanced bands; and
collapsing the plurality of enhanced bands to generate an enhanced image; and
displaying the enhanced image on a display.

9. The method of claim 8, further comprising:
capturing a video having a plurality of images; and
filtering each image of the plurality of images.

10. The method of claim 8, wherein the frequency filter is a temporal filter.

11. The method of claim 8, wherein the frequency filter is a color filter.

12. The method of claim 8, wherein a frequency of the frequency filter is set by a clinician.

13. The method of claim 8, wherein applying the frequency filter to the plurality of spatial frequency bands to generate the plurality of enhanced bands includes:
applying a color filter to the plurality of spatial frequency bands to generate a plurality of partially enhanced bands; and
applying a temporal filter to the plurality of partially enhanced bands to generate the plurality of enhanced bands.

14. The method of claim 13, wherein applying the color filter to the plurality of spatial frequency bands to generate the plurality of partially enhanced bands includes isolating an obstruction to a portion plurality of partially enhanced bands.

15. The method of claim 14, wherein applying the temporal filter to the plurality of partially enhanced bands to generate the plurality of enhanced bands includes applying the temporal filter only to the portion plurality of partially enhanced bands including the obstruction.
